(19) United States
(12) Patent Application Publication
(10) Pub. No.: US 2022/0110780 A1
(43) Pub. Date: Apr. 14, 2022

(54) PORTABLE URINAL DEVICE

(71) Applicant: Gaylene Ricketts, El Reno, OK (US)

(72) Inventor: Gaylene Ricketts, El Reno, OK (US)

(21) Appl. No.: 17/350,733

(22) Filed: Jun. 17, 2021

Related U.S. Application Data
(63) Continuation-in-part of application No. 17/070,105, filed on Oct. 14, 2020.
(60) Provisional application No. 63/132,931, filed on Dec. 31, 2020.
(60) Provisional application No. 63/132,973, filed on Dec. 31, 2020.
(60) Provisional application No. 63/132,980, filed on Dec. 31, 2020.

(51) Int. Cl. A61F 5/451; A61F 5/44

(52) U.S. Cl. CPC A61F 5/451; A61F 5/4556; A61F 5/4404

(57) ABSTRACT

The disclosure comprises a portable urinal device. The urinal device comprises a housing with a bracket arm movable from an open configuration to a closed configuration. The bracket arm is connectable to a waste assembly, which is configured to stay in a folded configuration when in the housing in the closed configuration, and to expand to an expanded configuration when the bracket is extended from the housing in the open configuration.

### CROSS REFERENCE TO RELATED APPLICATIONS

This application is a continuation-in-part of U.S. patent application Ser. No. 17/070,105 filed on Oct. 14, 2020, and claims the benefit of U.S. Provisional Patent Application No. 63/132,931 filed on Dec. 31, 2020, U.S. Provisional Patent Application No. 63/132,973 filed on Dec. 31, 2020, and U.S. Provisional Patent Application No. 63/132,980 filed on Dec. 31, 2020, all of which are hereby incorporated by reference.

### TECHNICAL FIELD

The present disclosure relates generally to the field of urinal devices, and more particularly, to systems and methods using a portable urinal device and managing disposal of the urinal device.

### BACKGROUND OF THE INVENTION

Urination is a necessary bodily function to relieve the body of wastes. Unfortunately, timing and accessibility of suitable facilities can be factors that can make the need to urinate extremely uncomfortable or inconvenient. Even when a public toilet is readily available, the user often encounters the problem of dirty and unhygienic public toilets; including feces, urine, blood-borne pathogens, pandemic diseases, and other viruses and germs. People often need to use a lot of toilet paper to wipe the toilet or hover over the toilet. Even then, a lot of female users do not want to sit on the toilet because it is dirty, and prefer to hold their urine for extended periods causing physical complications; damage to the pelvic girdle, pelvic muscle and wall, kidneys, bladder infections, joints, etc.

Those that have major medical ailments or are wheelchair bound have a need to access a toilet to void without ever leaving their chair or placing pressure on other organs or joints, which can cause further harm or discomfort to themselves.

Children also may have to deal with the above issues, especially when physical limitations prohibit them from urinating in a standard size adult toilet or urinal.

While attempts have been made in the past to provide urine collecting or directing devices, in general these devices have required a learning process and skill in handling for proper usage. These devices are typically bulky and, in some instances, require the user to assemble the funnel-like device from a flat blank which if not properly erected will tend to leak and will not achieve the desired result. Some of these devices have been designed for reuse. These reusable devices must therefore be cleaned after each use before being stored in a handbag or the like for subsequent use. Such devices are awkward, unsanitary and inconvenient to use, particularly after initial use whereupon cleaning the device is required; sometimes in the same public washbasin used for hands.

Thus, there exists a need for a solution that addresses the ability to use a toilet or urinal without actual contact while providing better health for the pelvic girdle, fewer bladder infections, strain on joints and muscles, and cleaner and more convenient options. For these and other reasons, an effective, easy-to-use, portable elimination device is desirable.

### SUMMARY OF THE INVENTION

The present disclosure meets the needs described above comprising a method and system for a portable urinal. In some embodiments, the portable urinal device comprises a housing and a bracket arm. The housing comprises an exterior surface that defines an interior cavity. The bracket arm is configured to be movable between an extended configuration and a stationary configuration. In the extended configuration, the bracket arm extends from the housing. The bracket arm is further configured to removably couple with a cartridge having a folded configuration and an expanded configuration. The housing is configured to contain the bracket arm when the bracket arm is in the stationary configuration, and the bracket arm extends from the housing when the bracket arm is in the extended configuration such that, when the cartridge is coupled with the bracket arm, the cartridge extends outward from the housing.

In other embodiments, a urinal device comprises a housing with an exterior surface that defines an interior cavity. The urinal device includes a first actuator oriented on the housing. The urinal device further comprises a bracket arm wherein the first actuator is configured to activate the bracket arm from a stationary configuration to an extended configuration. The urinal device also comprises a plurality of cartridges. Each cartridge comprises a bracket configured to removably couple to the bracket arm and to extend from the housing when coupled to the bracket arm and when the bracket arm is in the extended position. The bracket can translate vertically in the interior cavity based on activation by a second actuator. A conduit coupled individually to an associated bracket. The waste conduits are each configured to stay in a folded configuration while in the housing. The waste conduits are configured to expand to an expanded configuration when the associated bracket is extended from the interior cavity. Each of the plurality of cartridges can individually and sequentially couple with the bracket arm by the bracket coupling with the bracket arm.

In other embodiments, a urinal device comprises a bracket and a waste conduit. The bracket defines an orifice, wherein the bracket and orifice lie in a first plane. The waste conduit has a side wall and has a folded configuration and an expanded configuration, wherein when in the folded configuration the waste conduit extends substantially along a second plane which is parallel to the first plane, and when in the expanded configuration the side wall defines an upper opening with the upper opening being co-planar with the orifice and with the sidewall extending down from the orifice.

In yet another aspect some embodiments are a method for using the urinal device. The method comprises activating the urinal device from a stationary configuration to an extended configuration. Activating the urinal device to the extended configuration comprises extending a cartridge from an interior cavity of the urinal device. In the stationary configuration, a waste conduit of the cartridge is in a folded configuration, and in the open configuration, the waste conduit is in an expanded configuration. The method includes providing a disposable substance to the waste assembly. The method can further comprise detaching a portion of the waste assembly for disposal.

In view of the foregoing, it will be appreciated that the present disclosure provides a system and method for using a portable urinal. The specific systems and techniques for accomplishing the advantages described above will become apparent from the following detailed description of the embodiments and the appended drawings and claims.

### BRIEF DESCRIPTION OF THE DRAWINGS

FIG. 1 depicts an isometric view of an embodiment in a closed configuration.

FIG. 2 depicts an isometric view of the embodiment in FIG. 1 in an open configuration.

FIG. 3A depicts an isometric view of the embodiment in FIG. 1 in an extended configuration along with the cartridge having a container in the expanded configuration.

FIG. 3B depicts an isometric view of the embodiment in FIG.1 in an open configuration along with the cartridge having a funnel in the expanded configuration.

FIG. 4 depicts a partial isometric top view of the embodiment in FIG. 1 with a top portion of the housing removed.

FIG. 5 depicts a partial isometric bottom view of the embodiment in FIG. 1 with a bottom portion of the housing removed.

FIG. 6 depicts a partial isometric top view of a second embodiment with a top portion of the housing removed.

FIG. 7 depicts a partial isometric top view of the embodiment in FIG. 6 with a top portion of the housing removed.

FIG. 8 depicts a rear cross-sectional isometric view of the embodiment in FIG. 6.

FIG. 9 depicts an isometric view of a third embodiment in a closed configuration.

FIG. 10 depicts an isometric view of the embodiment in FIG. 9 in an open configuration.

FIG. 11 depicts a partial isometric top view of the embodiment in FIG. 9 with a top portion of the housing removed.

FIG. 12 depicts a partial isometric side view of the embodiment in FIG. 9 with a top portion of the housing removed.

FIG. 13 depicts an isometric top view of the embodiment in FIG. 9 with a top portion of the housing removed.

FIG. 14 depicts an isometric bottom view of the embodiment in FIG. 9 with a bottom portion of the housing removed.

FIG. 15 depicts an isometric top view of a fourth embodiment with a top portion of the housing removed.

FIG. 16 depicts an isometric view of the embodiment in FIG. 9 in an open configuration with a portion of the top cover of the housing removed.

FIG. 17 depicts an isometric top view of another embodiment of a urinal device.

FIG. 18 depicts an isometric bottom view of the embodiment of FIG. 17, with bottom access panels open.

FIG. 19 is a top view of a cartridge in the folded configuration.

### DETAILED DESCRIPTION

Illustrative embodiments of the disclosure will now be described more fully hereinafter with reference to the accompanying drawings, in which some, but not all embodiments of the disclosure are shown. The disclosure may be embodied in many different forms and should not be construed as limited to the embodiments set forth herein; rather, these embodiments are provided so that this disclosure will satisfy applicable legal requirements.

Whenever appropriate, terms used in the singular also will include the plural and vice versa. The use of “a” herein means “one or more” unless stated otherwise or where the use of “one or more” is clearly inappropriate. The use of “or” means “and/or” unless stated otherwise. The use of “comprise,” “comprises,” “comprising,” “include,” “includes,” and “including” are interchangeable and not intended to be limiting. The term “such as” also is not intended to be limiting. For example, the term “including” shall mean “including, but not limited to.”

The following description is provided as an enabling teaching of the disclosed articles, systems, and methods in their best, currently known embodiments. To this end, those skilled in the relevant art will recognize and appreciate that many changes can be made to the various aspects of the articles, systems, and methods described herein, while still obtaining the beneficial results of the disclosure. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof

As used throughout, the singular forms “a,” “an” and “the” include plural referents unless the context clearly dictates otherwise. Thus, for example, reference to “a gasket” can include two or more such gaskets unless the context indicates otherwise.

As used throughout, “substantially” with respect to a measure can refer to a range of values comprising +/−10 degrees. For example, substantially orthogonal, normal, or parallel can include embodiments, where the referenced components are oriented +/−10 degrees of being classified as orthogonal, normal, or parallel respectively.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, the terms “optional” or “optionally” mean that the subsequently described event or circumstance may or may not occur, and that the description includes instances where said event or circumstance occurs and instances where it does not.

The word “or” as used herein means any one member of a particular list and also includes any combination of members of that list.

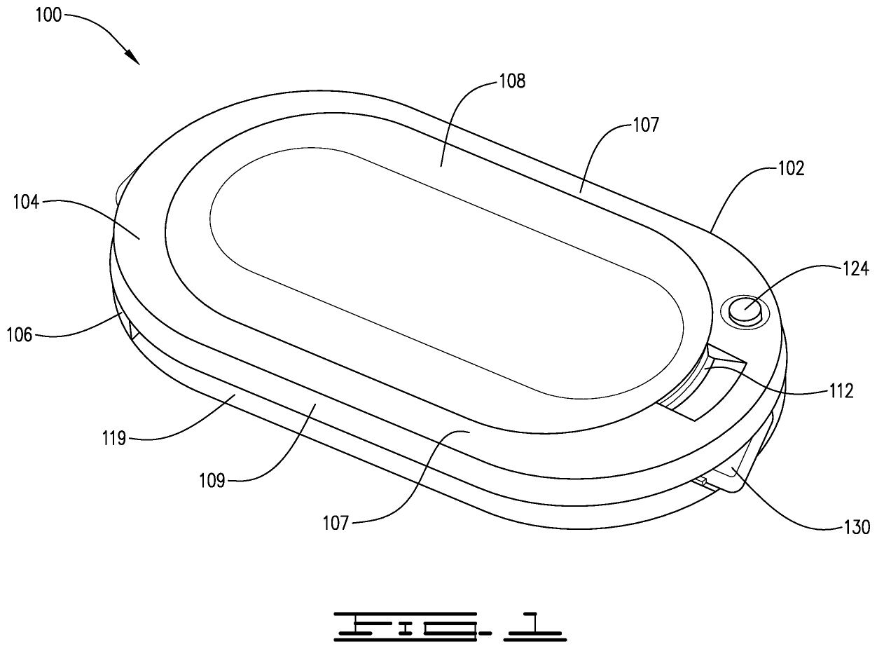

As shown in FIG. 1, the portable urinal system can be a urinal device 100. The urinal device 100 can comprise a housing 102. The housing 102 can comprise an exterior surface 108 that defines an interior cavity 103 (not shown). In a further aspect, the housing 102 can comprise a top portion 104 that mates with a bottom portion 106. The housing can also comprise a side surface 109. The side surface 109 can define a slit aperture 119 that serves as a passage for a waste assembly or cartridge 114 (not shown) to enter and exit the interior cavity of the housing 102. The housing 102 can be comprised of a metallic material. In another embodiment, the housing 102 can be comprised of rigid and/or semi rigid material such as a plastic. In another aspect, the exterior surface 108 can include gripping features and/or textured patterns 107. These gripping features can increase the tactile grip, which can reduce the urinal device 100 from slipping out of the user's hand in the event that the urinal device 100 gets wet. In a further aspect, the textured patterns can be etched patterns on the exterior surface 108. The gripping features can be portions of rubber or silicone adhered to the exterior surface to reduce slippage.

In a further aspect, the exterior surface 108 can comprise a top cover 110. The top cover 110 can be used to access the interior cavity 103 of the housing 102. In another aspect, the exterior surface 108 can define an access divot 112. The access divot 112 can be an indentation in the exterior surface 108. The access divot can be in proximity to the top cover 110. The access divot 112 can provide a user with additional dexterity in using their finger or tool to open the top cover 110 for gaining access to the interior cavity 103. In another embodiment, the access divot 112 can be defined in the top cover 110. The top cover 110 can also be made of transparent or semi-transparent material to permit the user to view into the interior cavity 103 to identify the contents of the interior cavity.

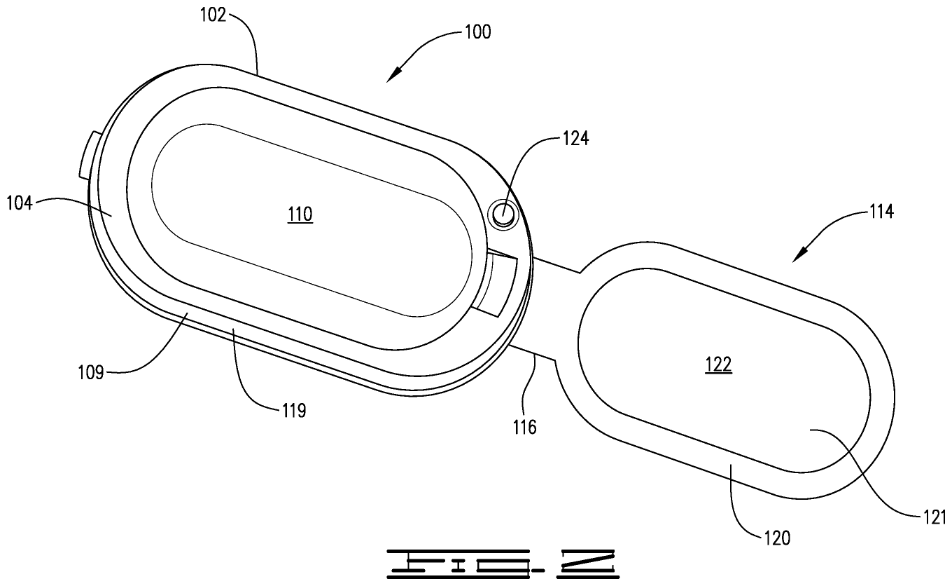

As shown in FIG. 2, the urinal device 100 can transition from the closed or stationary configuration in FIG. 1 to the open or extended configuration in FIG. 2. In the open or extended configuration, the waste assembly or cartridge 114 (herein waste assembly and cartridge are used interchangeably) extends from the interior cavity of the housing 102. In the closed or stationary configuration, the cartridge 114 resides in the interior cavity 103 of the housing 102.

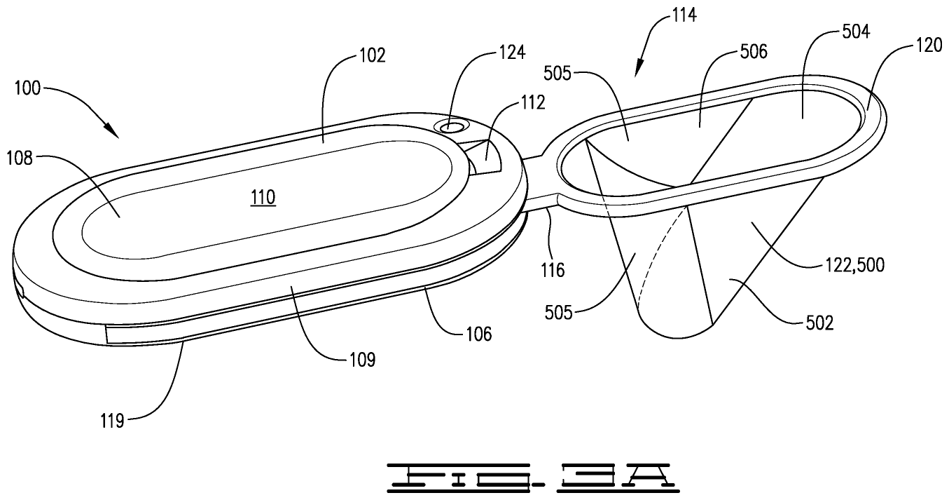

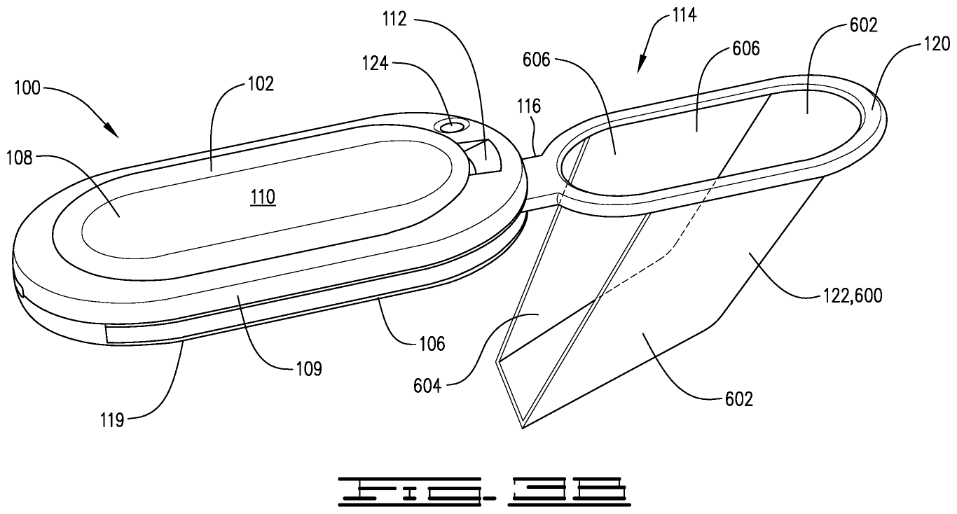

FIGS. 3A and 3B illustrate two embodiments of cartridge 114, each comprising a bracket 120 and a waste conduit 122, but differing in the design of the waste conduit 122, which will be described further below. In FIGS. 3A and 3B, the waste conduit 122 of the waste assembly 114 can transition from a folded configuration (see FIG. 19) to an expanded configuration (shown in FIGS. 3A and 3B). While in the folded configuration, the waste conduit 122 can have approximately the same dimensions of the bracket 120 to facilitate storage of the waste assembly 114 in the interior cavity 103.

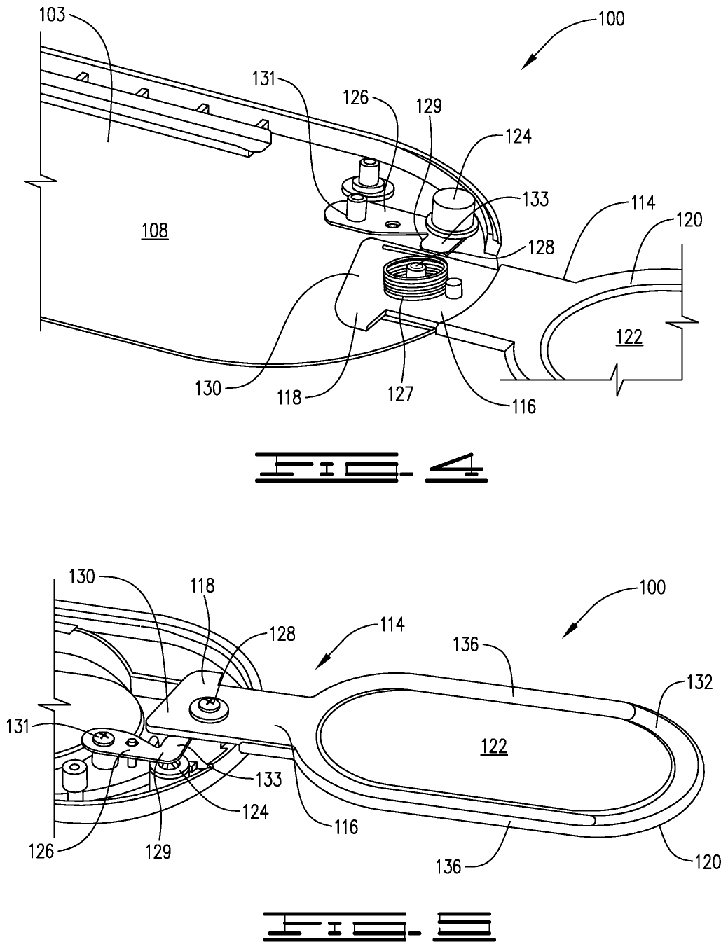

As shown in FIG. 4, the bracket 120 of cartridge 114 can be coupled to a bracket arm 116 of housing 102. The bracket arm 116 can comprise a member that is coupled to the bottom portion 106 of the housing 102. In another embodiment, the bracket arm 116 can be coupled to the top portion 104 (not shown). The bracket arm 116 can be configured to rotate around a pivot point 128. The bracket arm 116 can rotate around the pivot point 128 as the bracket arm 116 transitions the urinal device 100 from the open configuration to the closed configuration. Similarly, the waste assembly attached to bracket arm 116 can rotate around the pivot arm as the urinal device transitions from the open configuration to the closed configuration. In a further aspect, the bracket arm 116 can be spring loaded with a waste-assembly spring 127. The waste-assembly spring 127 can be a rotational spring with one end anchored to a portion of an interior surface of housing 102 and a second end of the rotational spring anchored to a surface of the bracket arm 116.

The bracket arm 116 can be activated to transition from a closed configuration to an open configuration with the use of a cartridge actuator 124. The cartridge actuator 124 can comprise a button or a cylindrical shaft. For example, the cartridge actuator 124 can be activated when the user applies a normal force relative to the housing by pressing the button; however, other arrangements are within the scope, such as a slide lever for activation. The button can be oriented to engage a free end 133 of the actuator hook member 126. The actuator hook member 126 can comprise a cantilevered member comprising a fixed end at fastener 131 and the free end 133. The free end 133 can further comprise a hook portion 129. In the closed configuration, the hook portion 129 at the free end 133 can be oriented to engage with a grip portion 118 of the bracket arm 116. The grip portion is on a region of the bracket arm that extends away from the surface of the bracket arm to serve as an engagement point. To transition from the closed configuration to the open configuration, the button of the actuator 124 can be pressed, which can force the free end 133 to flex vertically. The resultant action can cause the hook portion 129 of the hook member 126 to disengage the grip portion 118 of the bracket arm 116. When the engagement between the hook portion 129 and the grip portion 118 occurs, the bracket arm 116 can freely rotate to extend the cartridge 114 away from the interior cavity 103 of the housing 102, completing the transition to the open configuration. As discussed earlier, the urinal device 100 can include a waste-assembly spring 127. The transition from the closed configuration to the open configuration can be expedited by an embodiment with the waste-assembly spring 127. In this aspect, the waste-assembly spring 127 can be biased such that the potential energy is stored when the urinal device 100 is in the closed configuration. When the actuator 124 is activated to transition the urinal device from the closed configuration to the open configuration, the potential energy stored in the waste-assembly spring is released causing the bracket arm 116 to rotate swiftly from the interior cavity 103. In a further aspect, the bracket arm 116 can be comprised of a rigid or semi-rigid material such as a plastic or metal.

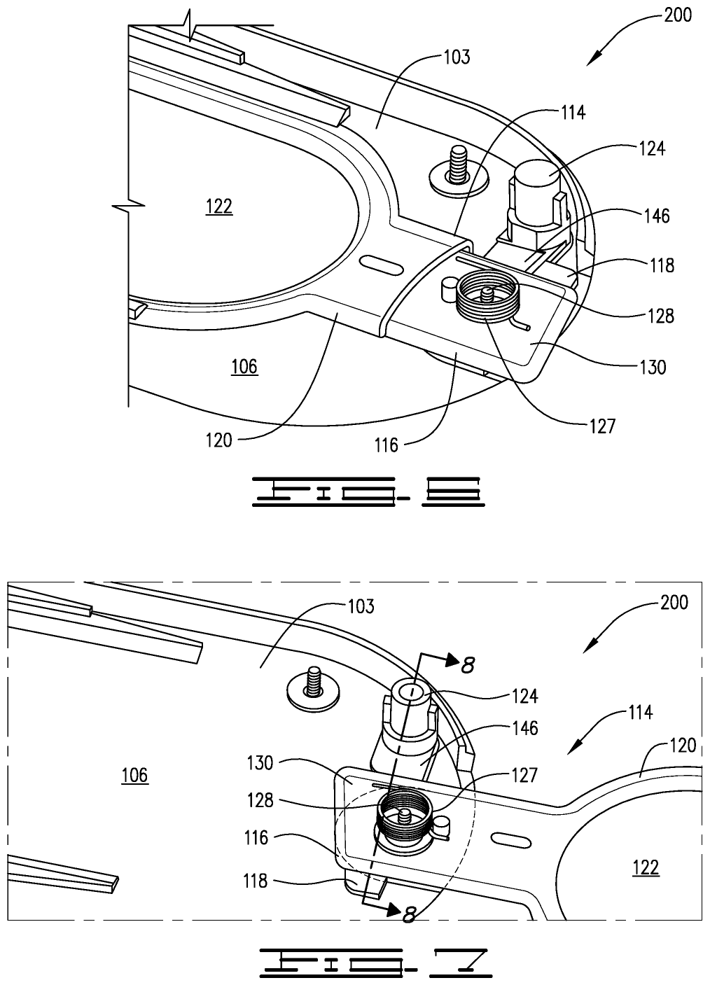

As shown in FIG. 5 and indicated above, the cartridge 114 can comprise a bracket 120. The bracket 120 can comprise a bottom surface that is configured in a channel configuration 132. The shape of the channel configuration 132 can be used as a fixture to provide engagement between the bracket 120 and the bracket arm 116. In a further aspect, the bracket arm 116 can have extender members 136. The extender members 136 can be shafts that protrude from a distal portion of the bracket arm 116. The extender members 136 can be used as an engagement surface between the bracket arm 116 and the bracket 120. The bracket 120 can engage the bracket arm 116 when the extender members 136 rest in the channel 132.

In a further aspect, the channel 132 of the bracket 120 can be comprised of a rigid or semi-rigid material such as a plastic or metal. Other embodiments of the bracket 120 can comprise combinations of polymers, rubber, or silicone. In a further aspect, the bracket 120 can be comprised of a corrugated cardboard to preserve some rigidity while providing increased biodegradability of the waste bracket for disposal. Similarly, the waste conduit 122 can be comprised of a biodegradable plastic, or wax-lined paper. In certain embodiments, the waste assembly 114 (including bracket 120 and waste conduit 122) will be made entirely of degradable material so that once detached from bracket arm 116, the waste assembly can be disposed of in an easy, safe and environmental manner. As used herein, degradable means biodegradable, compostable, and/or flushable. Biodegradable means a material breaking down into natural elements—typically carbon dioxide, water vapor and/or organic materials, which are not harmful to the environment—by organisms like bacteria and fungi. As used herein, biodegradable refers to a material in which 90% or more of the material breaks down within 1 year, and more preferably within 6 months or within 3 months. Compostable refers to a material, which similar to biodegradable materials, breakdown into natural elements but only in a compost setting. As used herein, compostable refers to material that break down in a compost setting within 1 year, and more preferably within 6 months or within 3 months. Flushable means a material that starts to breakdown in water so as dispersed in the water. Generally, the flushing action of a toilet will aid in the dispersion so that, if not prior to flushing, the breakdown will start immediately after flushing of the toilet. For example, the flushable material will be dispersed in water, so as not to clog plumbing, within 30 seconds, 20 seconds, or 15 seconds of flushing. For example, the waste conduit and/or the waste assembly can be made of soluble paper, poly-vinyl alcohol, cardboard shavings, and other water-soluble materials.

As shown in FIGS. 4 and 5, in a further aspect, the bracket arm 116 can further comprise an extender wedge 130. The extender wedge 130 can be a protrusion that extends from the surface of the bracket arm 116. When the user attempts to transition the waste assembly 114 from the open configuration to the closed configuration, the user can initially begin the rotation of the bracket arm 116 by applying a force to the extender members 136. After the extender members 136 have been returned to the interior cavity 103, the extender wedge 130 serves as a grip to allow the user to continually apply a force to the bracket arm, until the waste assembly 114 has been re-engaged into the closed position.

The bracket 120 can be coupled to a waste conduit 122. Waste conduit 122 can be better seen from FIG. 3A. FIG. 3A illustrates a waste conduit 122 that is in the form of a waste collector 500 having a side wall 502. Side wall 502 defines an opening 506 such that waste collector 500 has an open top but is otherwise enclosed in order to contain fluids and/or solids. As illustrated, side wall 502 has a slanted portion 504 as well as substantially vertical portions 505; thus, slanted portion 504 forms both a side wall and a bottom to container 500. However, other configurations are within the scope of the invention. Accordingly, waste collector 500 is in the form of a container that can hold waste until the user is ready to dispose of the waste. Waste can be introduced into waste collector 500 through opening 506 and be contained by side wall 502.

On the other hand, waste conduit 122 illustrated in FIG. 3B is in the form of waste funnel 600. Waste funnel 600 has a side wall 602 which defines a lower opening 604 and an upper opening 606. (As illustrated, lower opening 604 and upper opening 606 form a single opening spanning the lower portion and upper portions of funnel 600, but the opening can be separate, such as by having a short wall spanning the upper portion of opening 604.) Accordingly, waste funnel 600 is configured to channel waste that comes in through upper opening 606 towards and out through lower opening 604, which can be located at a suitable disposal receptacle for the waste. For example, upper opening 606 can be held on or close to the body so that waste can be received into funnel 600 through upper opening 606 and the waste is then channeled by funnel 600 to lower opening 604 which can be located at a toilet or urinal (not shown) such that waste exiting funnel 600 through lower opening 604 is received into the toilet or urinal.

In either embodiment, waste collector 500 or waste funnel 600, the waste conduit can be configured to have a folded configuration and an expanded configuration. FIGS. 3A and 3B show the expanded configuration for waste collector 500 and waste funnel 600, respectively. FIG. 19 illustrates the folded configuration for waste conduit 122, which is similar for both waste funnel 600 and waste collector 500.

The waste conduit 122 can be configured to be folded into the folded configuration when the cartridge 114 is in the interior cavity (closed configuration). When the cartridge 114 is extended into the open configuration, the waste conduit can be placed in the extended configuration. In some embodiments, the waste conduit 122 will automatically expand to the extended configuration as the waste assembly 114 is moved to the open configuration. However, generally the waste conduit 122 will be retained in the folded configuration during movement to the open configuration and even thereafter until the user is ready for the waste conduit 122 to be placed in expanded configuration. For example, a flap 410 can be used to retain waste conduit 122 in the folded configuration. Once flap 410 is folded back from waste conduit 122, waste conduit is free to move to the expanded configuration.

As can be seen from FIG. 2, bracket 120 defines an orifice 121. Bracket 120 and orifice 121 lie in a first plane. When in the folded configuration, waste conduit 122 extends substantially along a second plane which is in and/or parallel to the first plane—the plane containing bracket 120 and orifice 121. When in the expanded configuration as shown in FIGS. 3A and 3B, side wall 502 or 602 defines an upper opening 506 and 606, respectively, with the upper opening being co-planar with orifice 121 and with the sidewall extending down from the orifice.

Once in the expanded configuration, the user has access to the interior of the waste conduit 122 of the waste assembly 114. The user can then provide fluids or substances into the waste conduit for their desired purpose (i.e. urination). The waste conduit 122 can be comprised of a degradable material, as described above. In some instances, the waste conduit 122 will expand upon release of flap 410 (shown in FIG. 19). In other instances, the weight liquids and/or substances can cause the waste conduit 122 to transition from a folded configuration to a container configuration. In still other instances, the user can aid the transition by manually pressing the waste conduit surface in the folded configuration or by flicking the waste assembly to open the waste conduit. When the user completes using the urinal device 100, the user can disengage the bracket 120 (including the waste conduit 122) from the bracket arm 116. The user can then discard the waste conduit 122 and bracket 120. For example, where a flushable material is used, the waste assembly can be dropped in a toilet and flushed away.

As will be realized from the previous paragraph, in one aspect the bracket 120 is removably coupled to the bracket arm 116. The removably coupled configuration permits the user to disengage the bracket 120 from bracket arm 116, including the extender members 136 if used. In an alternate embodiment, the cartridge 114 can be configured such that the only removable/discarded portion would be the waste conduit 122. For example, after use to either collect removable substances, either liquid or solid, or to channel removable substance to a disposal device, only the waste conduit 122 is removed from the waste assembly 114. In other embodiments, the entire urinal device 100 can be disposable, including housing 102, bracket arm 116, bracket 120 and waste conduit 122.

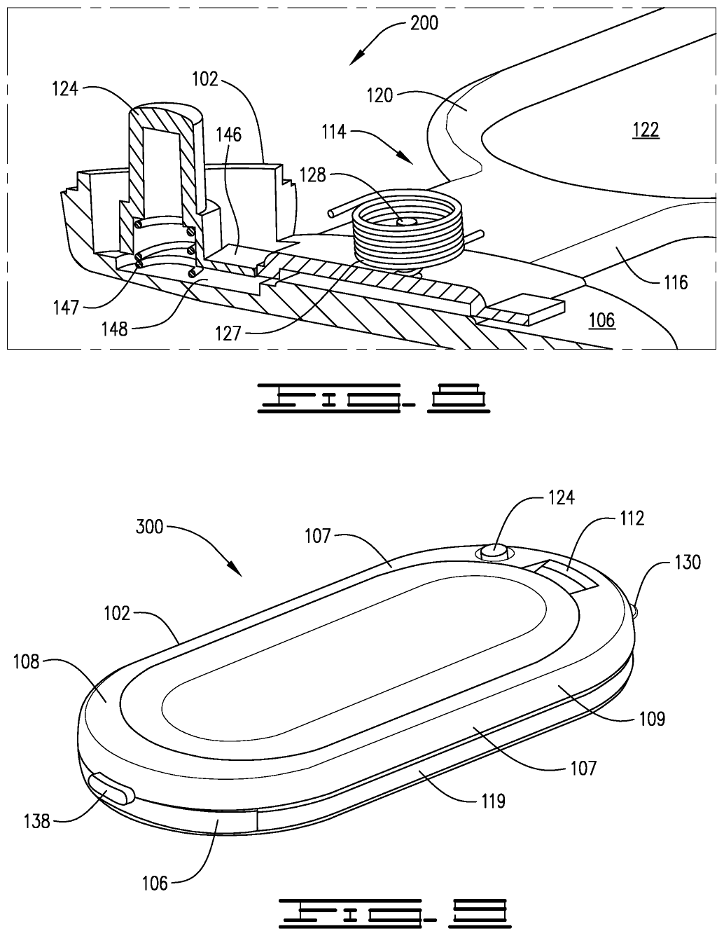

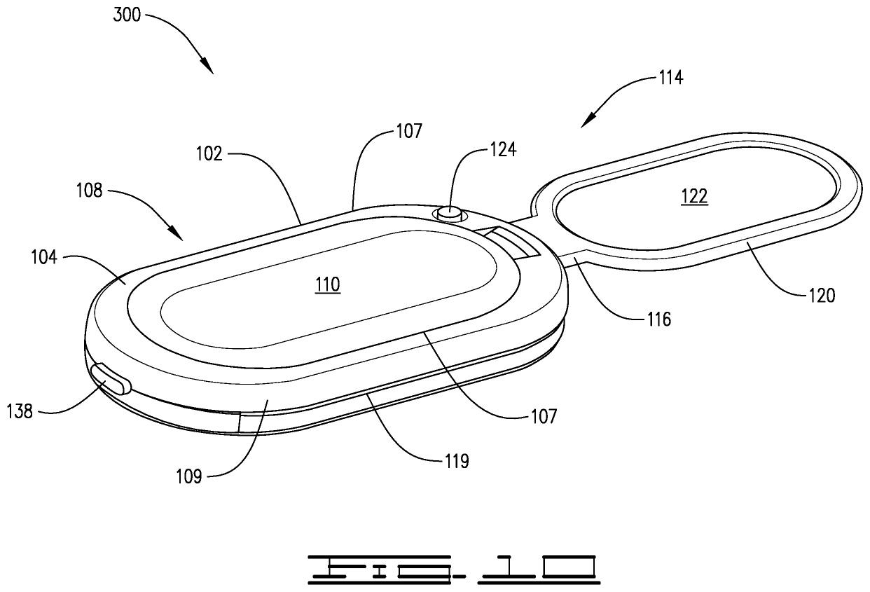

As shown in FIGS. 6, 7, and 8, urinal device 200 can comprise another embodiment for the waste-assembly actuator 124. The waste-assembly actuator 124 can comprise a button or a cylindrical shaft. Towards the base of the cylindrical shaft can be a flange 146 that extends from the base. As shown in FIG. 6, urinal device 200 is in the closed configuration. In the closed configuration, the flange 146 is engaged to grip portion 118 of the bracket arm 116, which prevents the bracket arm from rotating around the pivot point 128. As shown in FIGS. 7 and 8, urinal device 200 is in an open configuration. To transition to the open configuration, a user can apply a normal force to the waste-assembly actuator 124. As shown in FIG. 8, the waste-assembly actuator 124 can comprise a compression spring 147. When the normal force is applied, the waste-assembly actuator 124 can be lowered into the recess 148. The recess 148 can be a divot in the bottom portion 106 of the housing interior. When waste-assembly actuator 124 is lowered into the recess 148, the flange 146 can no longer engage the grip portion 118 of the bracket arm 116. The bracket arm 116 can then be free to rotate around the pivot point 128. When the actuator 124 is no longer pressed, the potential energy stored in the compression spring 147 can be released, which elevates the waste-assembly actuator 124 from the recess 148 back to a resting position.

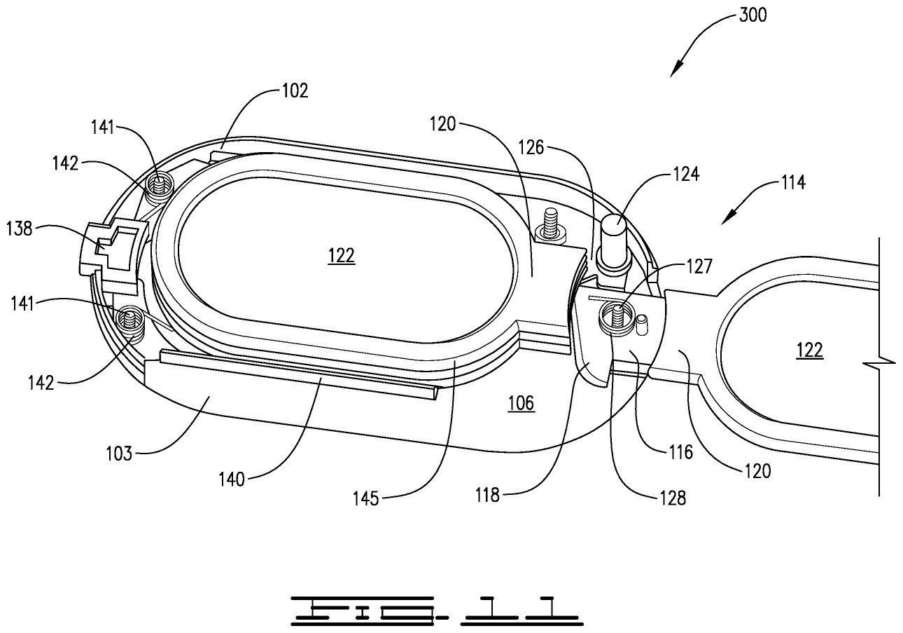

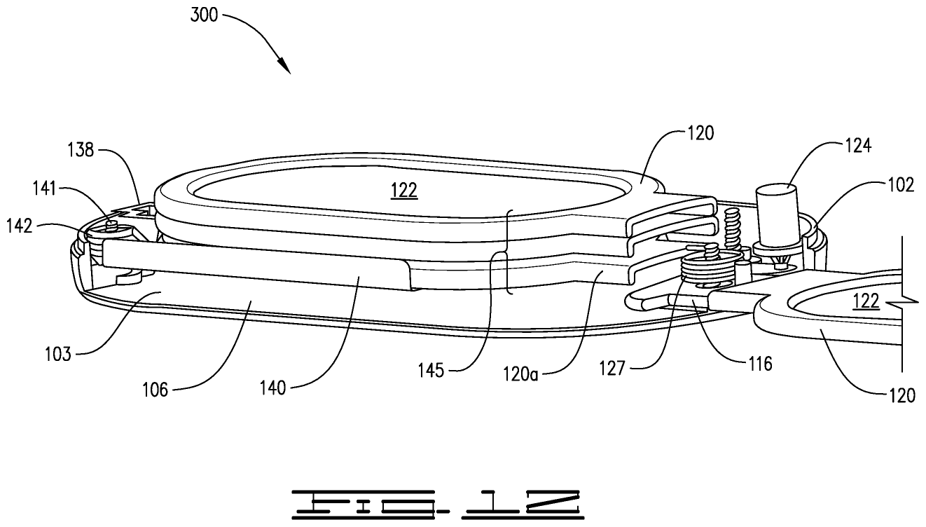

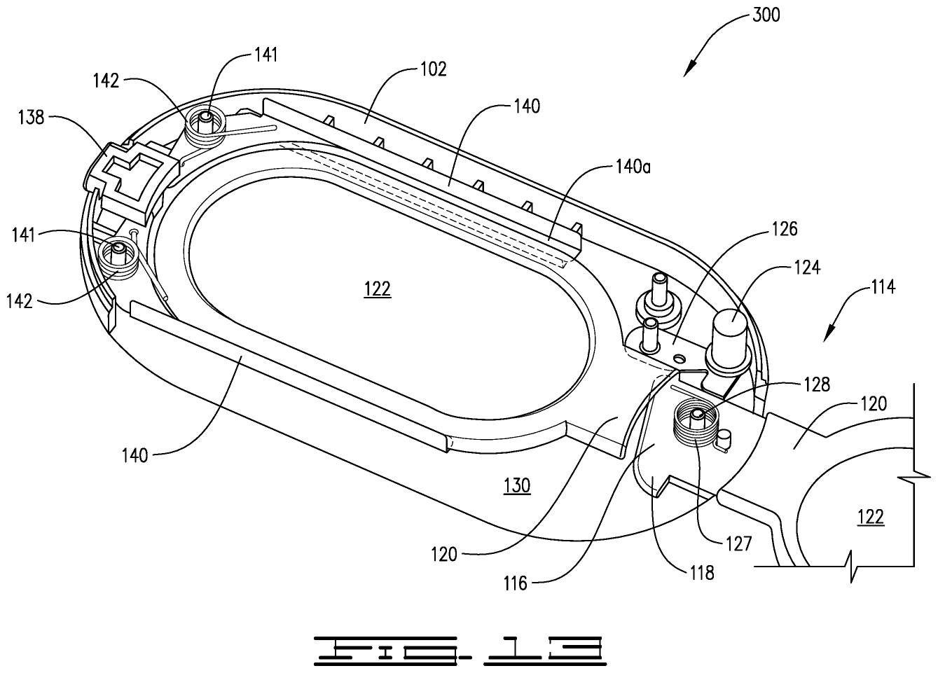

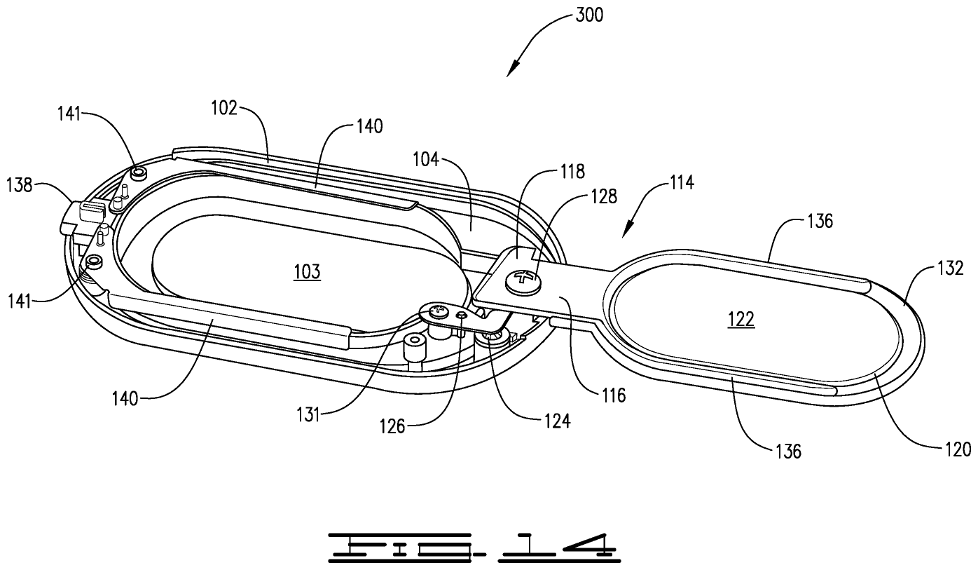

In FIG. 9 and FIG. 10, the urinal device 300 can comprise multiple removable brackets 120. The urinal device 300 shown in the closed configuration in FIG. 9 and open configuration in FIG. 10 both further comprise a second actuator, the bracket release actuator 138. As shown in FIG. 11 and FIG. 12, multiple cartridges 114 can be stacked on top of each other to form a stack 145 of cartridges 114. The stack 145 of cartridges 114, each with a bracket 120 and associated waste conduits 122, can be a method for packaging multiple brackets and waste conduits, which also facilitates ease of refilling the urinal device 300. In order to transition between the multiple brackets in a stack, urinal device 300 can utilize the bracket release actuator 138.

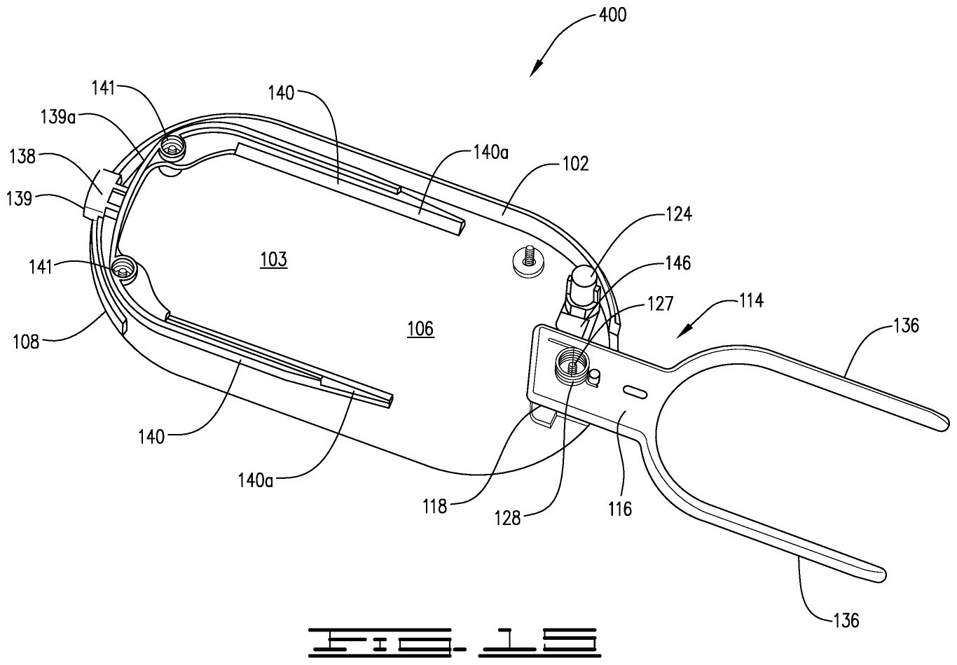

As shown in FIG. 13 and FIG. 14, the bracket release actuator 138 can be a button that when pressed activates the movement of release members 140. The release members 140 can be a set of shafts or rods coupled to the bracket release actuator 138. The release members 140 can be used to engage the stack of brackets 120. When the bracket release actuator 138 is pressed, the coupling between the release members 140 can cause an end of the release members to rotate outward away from the center of the brackets 120. The release members 140 can rotate around the auxiliary pivot points 141. As the release members 140 rotate around their respective auxiliary pivot points 141, the release members 140 can release or disengage from the bracket 120. As the release members 140 disengage from the bracket 120, the stack 145 of brackets can translate vertically from a stationary position in an upper region of the interior cavity 103 to the bottom portion 106 of the housing 102. The lowest bracket 120a (shown in FIG. 12) can then engage the bracket arm 116 as the channel 132 of the lowest bracket 120a comes to rest on the extender members 136 of the bracket arm. In a further aspect, the release members 140 can be coupled to release springs 142. The release springs 142 can be biased to hold the release members 140 inward to engage the bracket 120/stack 145 of brackets, holding the bracket 120/stack 145 of brackets in a stationary position. The release members 140 can have a surface 140a configured to engage the bracket 120. In this manner, each of the plurality of cartridges can individually and sequentially couple with the bracket arm 116.

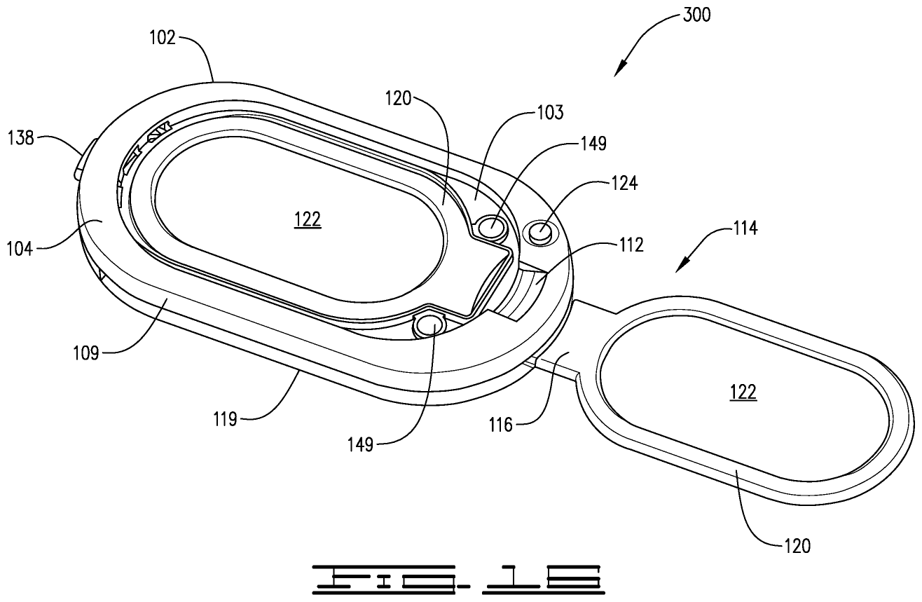

As shown in FIG. 15, the urinal device 400 differs from the urinal device 300 in that the bracket release actuator 138 in urinal device 400 can comprise a unitary piece. The unitary formation of the bracket release actuator 138 can comprise subcomponents. In urinal device 400, the subcomponents of the bracket release actuator 138 can comprise a protrusion 139, a crossbeam member 139a, and release members 140. The protrusion 139 can extend from the exterior surface 108 to serve as a point where is applied. Similar to urinal device 300, the release members 140 can have a surface 140a configured to engage the bracket 120. As a unitary piece, the bracket release actuator 138 will flex when the protrusion 139 is pressed inward towards the interior cavity 103 of the housing 102. The protrusion 139 is coupled to a crossbeam member 139a; the crossbeam member 139a is connected to both of the release members 140. The deflection of the crossbeam member 139a can cause the release members 140 to rotate around the auxiliary pivot points 141. The rotating release members 140 can result in either engaging or disengaging the bracket 120 (not shown) from a stationary position. In one aspect, the release actuator can be comprised of a thin metal to permit the necessary flexion and in another aspect the release actuator can comprise a flexible plastic.

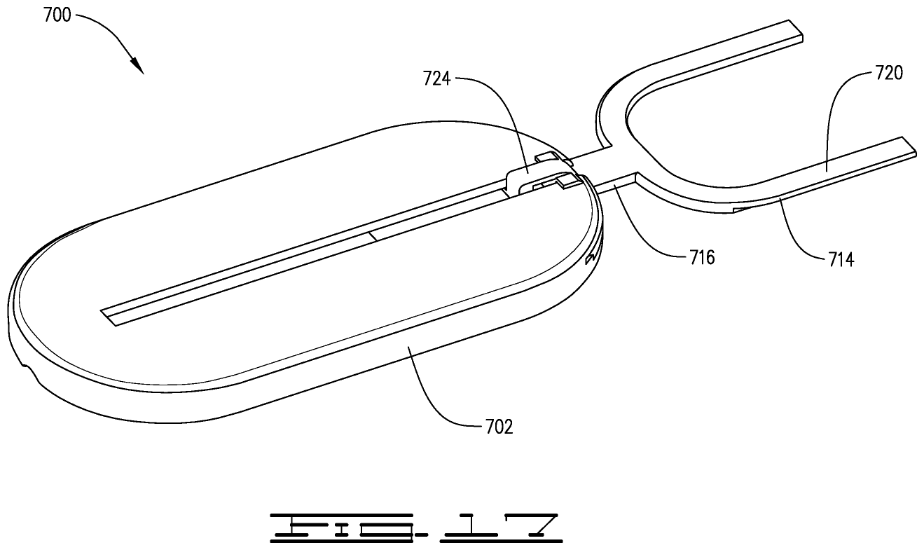

As shown in FIG. 16, top cover 110 can be removed to provide access to the multiple brackets 120 in the interior cavity 103. In one aspect, a portion of the top cover 110 (not shown) can comprise metallic components and/or a magnet. The metallic components and/or a magnet components of the top cover 110 can be oriented to mate with the housing magnets 149 oriented in the top portion 104 of the housing 102. The magnetic engagement between the top cover 110 and top region of the housing 102 can secure a seal between the top cover 110 and interior cavity 103. The user can still disengage the top cover 110 via the access divot 112. In another aspect, the top cover and the top portion 104 can be secured with a snap fit engagement. In a further aspect, a portion of the top cover 110 can comprise transparent or semi-transparent material, which would allow the user to identify how many cartridges are left.

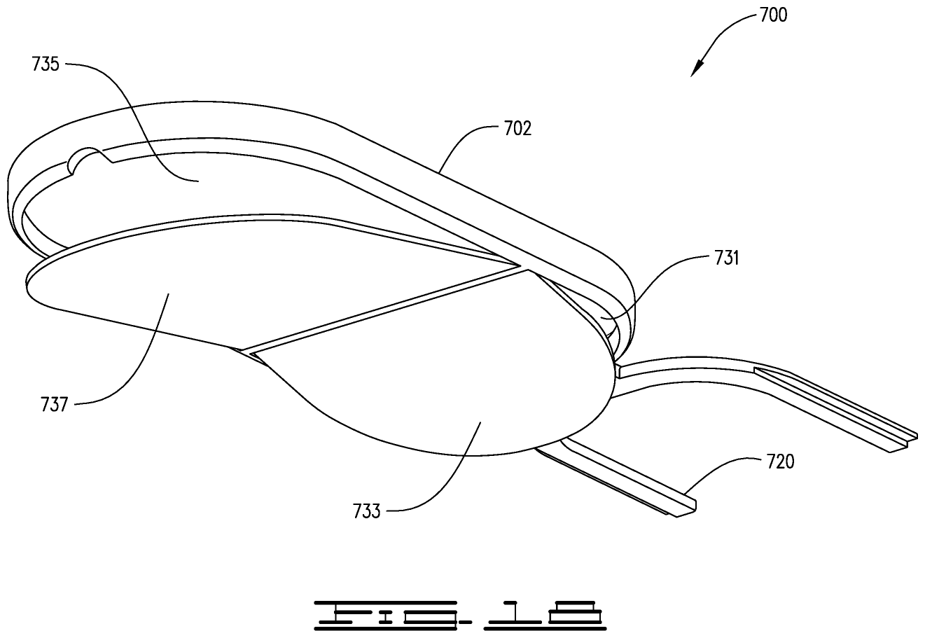

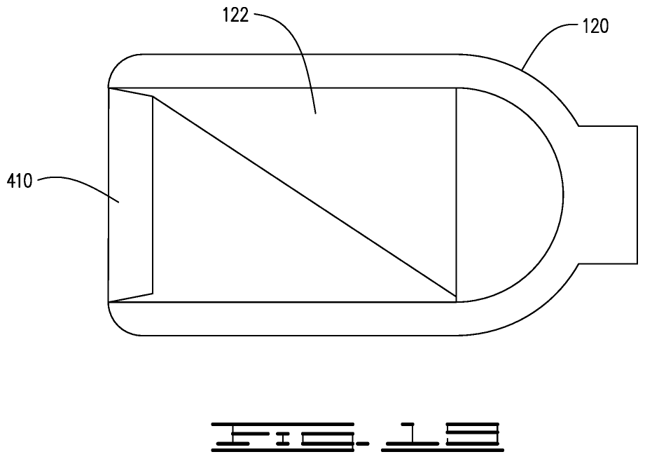

FIGS. 17 and 18 show another embodiment of the portable urinal system. These figures illustrate a urinal device 700, which is similar to urinal device 100 and 300, as described above, but is designed to slide the bracket arm 716 out from a housing 702 longitudinally or along the long axis of housing 702. An actuator 724 is provided, which facilitates movement of bracket arm 716 from the interior of the housing (the stationary configuration) out to the extended configuration shown in FIGS. 17 and 19. As will be realized, when bracket arm 716 is slid to the extended configuration, it can carry a cartridge 714 (here simply illustrated as U-shaped bracket 720, but which normally would have the bracket and a waste conduit, as previously described). While in some embodiments, cartridge 714 will be added after bracket arm 716 is slid into the extended configuration, generally one or more cartridges 714 will be stored in a cartridge storage space 731, which can be accessed by opening a first panel 733. If more than one cartridge is stored in cartridge storage space 731, it will be in a stack as described above, which can operate similar to the stack for urinary device 300, accounting for linear movement rather that pivotal movement. Additionally, housing 702 can have a second access panel 737, which allows access to the bracket arm when in the stationary or closed configuration and/or allows access to a space 735 that can be used to store items, such as hand wipes.

While the disclosure has been described in connection with what is presently considered to be the most practical and various embodiments, it is to be understood that the disclosure is not to be limited to the disclosed embodiments, but on the contrary, is intended to cover various modifications and equivalent arrangements comprised within the spirit and scope of the appended claims.

What is claimed is:

1. A urinal device comprising:
a housing comprising an exterior surface that defines an interior cavity; and
a bracket arm movable between an extended configuration and a stationary configuration, wherein the bracket arm is configured to removably couple with a cartridge having a folded configuration and an expanded configuration, and wherein the housing is configured to contain the bracket arm when the bracket arm is in the stationary configuration, and the bracket arm extends from the housing when the bracket arm is in the extended configuration such that, when the cartridge is coupled with the bracket arm, the cartridge extends outward from the housing when the bracket arm is in the extended configuration.

2. The urinal device of claim 1, further comprising an actuator oriented on the exterior surface of the housing, the actuator being configured to activate the bracket arm to move from the closed configuration to the open configuration.

3. The urinal device of claim 1, wherein the bracket arm is configured to rotate around a pivot point as the bracket arm extends from the housing into the extended configuration, and the waste assembly is configured to return to the stationary configuration by rotating around the pivot point.

4. The urinal device of claim 1, wherein the bracket arm is configured to slide longitudinally with respect to the housing to move between the extended configuration and the stationary configuration.

5. The urinal device of claim 1, further comprising the cartridge removably coupled to the bracket arm and having waste conduit configured to stay in the folded configuration while the cartridge is in the housing when the bracket arm is in the stationary configuration, and configured to extend from the housing and expand to the expanded configuration when the bracket arm is extended from the housing into the extended configuration.

6. The urinal device of claim 5, wherein the cartridge has a flap which retains the waste conduit in the folded configuration until the flap is moved to a release position.

7. The urinal device of claim 5, wherein the cartridge is made of a degradable material.

8. The urinal device of claim 5, wherein the waste conduit is a container having an open top and configured to hold waste.

9. The urinal device of claim 5, wherein the waste conduit is a funnel having an open top and an open bottom and configured to channel waste away from a user.

10. A urinal device comprising:
a housing comprising an exterior surface that defines an interior cavity;
a first actuator oriented on the exterior surface of the housing;
a bracket arm, wherein the first actuator being configured to activate the bracket arm from a stationary position to an extended position,
a plurality of cartridges, each cartridge comprising:
a bracket configured to removably couple to the bracket arm and to extend from the housing when coupled to the bracket arm and when the bracket arm is in the extended position, and
a waste conduit coupled to the bracket and each configured to stay in a folded configuration while in the housing and each of the plurality of waste conduits configured to individually expand to an expanded configuration when the associated bracket is extended from the housing;
wherein each of the plurality of cartridges can individually and sequentially couple with the bracket arm by the bracket coupling with the bracket arm.

11. The urinal device of claim 10, further comprising a second actuator oriented on the exterior surface of the housing, wherein the plurality of brackets translates vertically in the interior cavity based on activation by the second actuator.

12. The urinal device of claim 10, wherein the cartridge has a flap which retains the waste conduit in the folded configuration until the flap is moved to a release position.

13. The urinal device of claim 10, wherein the cartridge is made of a degradable material.

14. The urinal device of claim 10, wherein the waste conduit is a container having an open top and configured to hold waste.

15. The urinal device of claim 10, wherein the waste conduit is a funnel having an open top and an open bottom and configured to channel waste away from a user.

16. The urinal device of claim 10, wherein the bracket arm is configured to rotate around a pivot point to move to the extended configuration so that the waste assembly extends from the housing, and the bracket arm is configured to return to the stationary configuration by rotating around the pivot point.

17. The urinal device of claim 10, wherein the first actuator is configured to move the bracket arm longitudinally with respect to the housing so that the waste assembly extends from the housing in the extended position, and wherein the first actuator is configured to return the bracket arm to the stationary position.

18. A method for using a urinal device comprising:
activating a urinal device from a stationary configuration to an extended configuration, wherein activating the extended configuration comprises extending a cartridge from an interior cavity of the urinal device, wherein in the stationary configuration a waste conduit of the cartridge is in a folded configuration and in the extended configuration the waste conduit is in an expanded configuration;
providing a disposable substance to the waste conduit; and
detaching the cartridge for disposal.

19. The method of claim 18, further comprising:
returning the urinal device from the extended configuration to the stationary configuration;
positioning a new cartridge in the interior cavity of the urinal device; and
reactivating the urinal device from the stationary configuration to the extended configuration with the new cartridge moving to the expanded configuration once the urinal device is in the extended configuration.

20. The method of claim 18, wherein the waste assembly is made of a degradable material.

21. The method of claim 18, wherein the waste conduit is a container having an open top and configured to hold waste.

22. The method of claim 18, wherein the waste conduit is a funnel having an open top and an open bottom and configured to channel waste away from a user and wherein the method further comprises channeling the waste provided to the waste conduit to a waste disposal unit.
